(19) United States
(12) Patent Application Publication
(10) Pub. No.: US 2008/0113924 A1
(43) Pub. Date: May 15, 2008

(54) Neuramindase Inhibitor

(75) Inventors: Shinichiro Nishimura, Hokkaido (JP); Hiroshi Hinou, Hokkaido (JP); Hirosato Kondo, Osaka (JP); Kazuhiko Fujiwara, Tokyo (JP)

Correspondence Address: FOLEY AND LARDNER LLP;SUITE 500, 3000 K STREET NW, WASHINGTON, DC, 20007, US

(73) Assignees: NATIONAL INSTITUE OF ADVANCE INDUSTRIAL SCIENCE AND TECHNOLOGY, TOKYO, JAPAN (JP); NATIONAL UNIVERSITY CORPORATION HOKKAIDO UNIVERSITY, HOKKAIDO JAPAN (JP); SUMITOMO BAKELITE CO., LTD., TOKYO, JAPAN (JP)

(21) Appl. No.: 11/721,581

(22) PCT Filed: Dec. 16, 2005
(86) PCT No.: PCT/JP05/23162

§ 371 Date: Aug. 24, 2007

(30) Foreign Application Priority Data
Dec. 17, 2004 (JP) 2004-365660

(51) Int. Cl. A61K 31/7048; A61K 31/366; A61K 31/35; C07D 311/02

(52) U.S. Cl. 514/25; 514/27; 536/18.7; 514/457; 514/459; 549/283; 549/449

(57) ABSTRACT

There are provided a novel compound having irreversible inhibitory activity against neuraminidase, a therapeutic agent and a detection agent for a disease involving neuraminidase. A compound represented by the following formula (I) and a salt thereof, a production method thereof, and an application method thereof,

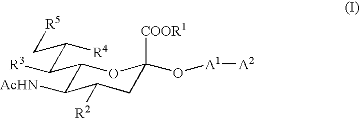

wherein:
A¹ represents an aryl group optionally having a substituent group or a heteroaryl group optionally having a substituent group;
A² represents —CX₂R⁶ or —CHXR⁶ wherein X represents —F, —Cl, —Br, or —I;
R¹ represents a hydrogen atom or an alkyl group optionally having a substituent group;
R², R³, R⁴, and R⁵ represent each independently —OC(═O)R⁶, —OR⁶, —N(R⁶)₂, —N₃, —NHC(═NH)NHR⁶, —NHCOR⁶, —OSO₃R⁶, —OPO₃(R⁶)₂, F, Cl, Br, or I; and
R⁶ represents each independently a hydrogen atom, an alkyl group optionally having a substituent group, an aryl group optionally having a substituent group, or an optionally substituted heteroaryl group.

### TECHNICAL FIELD

The present invention relates to a novel compound group having irreversible inhibitory activity against neuraminidase, and medical and biochemical applications thereof. More particularly, the present invention relates to a novel glycoside derivative of α-D-neuraminic acid, a production method thereof, and medical and biochemical applications thereof.

### BACKGROUND ART

Enzymes with the ability to cleave N-acetyl neuraminic acid (NANA), also known as sialic acid, from other sugars are present in many microorganisms. Examples of these include: bacteria such as Vibrio cholerae, Bacillus anthracis, Salmonella enterica, Clostridium perfringe, gas bacillus, Streptococcus pneumoniae, Bordetella parapertussis, syphilis bacterium, Francisella tularensis, Pseudomonas aeruginosa, Streptococcus mutans, Propionibacterium acnes, Helicobacter pylori, and Arthrobacter sialophilus; parasites such as Trypanosoma cruzi, Trypanosoma brucei, Leishmania major, and Plasmodium chabaudi; viruses such as influenza virus, avian influenza virus, parainfluenza virus, measles virus, mumps virus, Sendai virus, and Newcastle disease virus.

Many of these neuraminidase-possessing microorganisms are major pathogens of human and/or animals. In addition, many infection cases of influenza virus, measles virus, Streptococcus pneumoniae, Vibrio cholerae, Plasmodium chabaudi, Leishmania major, and trypanosomes are reported every year and cause enormous damages (Non-patent document 1).

It has been thought that inhibitors of neuraminidase activity might prevent infection or disease state by viruses, etc. possessing neuraminidase from becoming serious. Based on such thought, various neuraminidase inhibitors have been developed. Most of the known neuraminidase inhibitors are analogues of 2-deoxy-2,3-dehydro-N-acetylneuraminic acid (DANA) and its derivatives (Non-patent document 2, Patent documents 1 and 2). Patent document 1 also describes that 4-guanidino-2-deoxy-2,3-dehydro-N-acetylneuraminic acid is effective in treating influenza A virus and influenza B virus.

On the other hand, most of these neuraminidase inhibitors are competitive inhibitors. Only two cases have been reported regarding irreversible inhibitors having covalent bond formation ability with neuraminidase, but neither case considers molecular label associated with bond formation or isolation of neuraminidase (Non-patent documents 3 and 4).

Further, fluorinated glucose is mainly used as an irreversible inhibitor against glycolytic enzyme, and also Kurogochi et al., Ichikawa et al., or C-H. Wong et al. have reported labeling agents (Non-patent documents 5, 6, and 7). However, an irreversible inhibitor labelable against neuraminidase has not been developed yet.

On the other hand, α,-D-neuraminic acid having a glycosidic bond via a phenolic hydroxyl group is a good substrate for neuraminidase, and thus p-nitrophenyl glycoside (Patent Document 3), 4-methylumbelliferyl glycoside (Non-Patent Document 8), or the like has been used for many years as a good substrate for neuraminidase detection or activity measurement. Further, derivatives that give a color upon cleavage of sialoside bond have been reported (Patent Documents 4 and 5), but they do not have a neuraminidase inhibitory ability and they cannot directly label or isolate neuraminidase. As described above, almost no reports are found on compounds that can label neuraminidase itself for isolation and detection (Non-Patent Document 9).
Patent Document 1: JP Patent Publication (Kokai) No. 6-25209 A (1994)
Patent Document 2: JP Patent Publication (Kokai) No. 2-88594 A (1990)
Patent Document 3: JP Patent Publication (Kokai) No. 5-339283 A (1993)
Patent Document 4: JP Patent Publication (Kokai) No. 2001-131074 A
Patent Document 5: PCT/US98/22786
Non-Patent Document 1: G. Taylor, Current Opinion in Structural Biology, 6, 830-837 (1996)
Non-Patent Document 2: P. Meindl et al, Virology, 58, 457-463 (1974)
Non-Patent Document 3: P.-A. Driguez et al, Bioorg. Med. Chem. Lett, 2, 1361-1366 (1992)
Non-Patent Document 4: B. Barrere et al, Arch. Virol., 142, 1365-1380 (1997)
Non-Patent Document 5: M. Kurogchi et al, J. Biol. Chem., 43, 44704-44712 (2004)
Non-Patent Document 6: C.-H. Wong et al, Science, 275, 945-948 (1997)
Non-Patent Document 7: Y. Ichikawa et al, Bioorg. Med. Chem. Lett, 11, 1769-1773 (2001)
Non-Patent Document 8: M. Potier et al, Anal. Biochem., 94, 287 (1979)
Non-Patent Document 9: H. Hinou et al, Biochemistry, 44, 11669-11675 (2005)

An object of the present invention is to provide a novel compound having an irreversible inhibitory activity against neuraminidase and a therapeutic agent for diseases involving neuraminidase.

### DISCLOSURE OF THE INVENTION

The present inventors have made intensive study to solve the above problem. As a result, they have found that a compound having a specific electrophilic functional group introduced to an aglycone site of sialic acid enables the functional group upon cleavage of glycoside bond to work as an electrophilic agent, and this forms a covalent bond with a nucleophilic functional group near an active site of neuraminidase, thereby inhibiting its enzymatic function. Further, they have found that binding of further a substituent group, a chromophore, a fluorescent group, or a solid-phase carrier to this electrophilic functional group enables the controlling of neuraminidase inhibitory ability, rapid detection of neuraminidase, and capture, concentration, isolation and analysis of neuraminidase. Furthermore, they have found that binding of a drug such as antibiotic, an antibacterial metal fine particle, or a polyfunctional polymer to the electrophilic functional group enables addition of a function of DDS (drug delivery system). Based on these findings, the present inventors have completed the present invention.

Namely, the present invention includes the following inventions.
(1) A compound represented by the following formula (I) or a salt thereof, or a hydrate thereof,

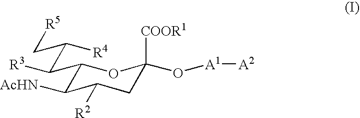

wherein:
A¹ represents an aryl group optionally having a substituent group or a heteroaryl group optionally having a substituent group;
A² represents —CX₂R⁶ or —CHXR⁶ wherein X represents —F, —Cl, Br, or —I;
R¹ represents a hydrogen atom or an alkyl group optionally having a substituent group;
R², R³, R⁴, and R⁵ represent each independently —OC(═O)R⁶, —OR⁶, —N(R⁶)₂, —N₃, —NHC(═NH)NHR⁶, —NHCOR⁶, —OSO₃R⁶, —OPO₃(R⁶)₂, F, Cl, Br, or I; and
R⁶ represents each independently a hydrogen atom, an alkyl group optionally having a substituent group, an aryl group optionally having a substituent group, or an optionally substituted heteroaryl group.
(2) A compound represented by the following formula (II) or a salt thereof, or a hydrate thereof,

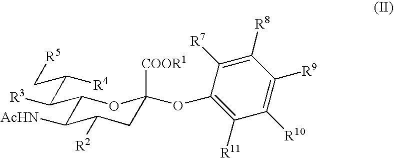

wherein:
R¹, R², R³, R⁴, and R⁵ are the same as defined in (1);
R⁷, R⁸, R⁹, R¹⁰, and R¹¹ represent each independently a hydrogen atom, —NO₂, —F, —Cl, —Br, —I, —N₃, —CN, —Ph, —CX₂R¹², —CHXR¹², —(CH₂)ₘR¹², —CR¹²═CR^{12′}—R^{12″}, —C≡C—R¹², —OP(═O)R¹²(OR^{12′}), —P(═O)R¹²(OR^{12′}), —OPR¹²(OR^{12′}), —PR¹²(OR^{12′}), —OP(R¹²)₂, —P(R¹²)₂, —OS(═)₂R¹², —S(═O)₂R¹², —OS(═O)R¹², —S(═O)R¹², —C(═O)R¹², or —NR¹²C(═O)R^{12′} (wherein at least one of R⁷, R⁹, and R¹¹ represents each independently —CX₂R¹² or —CHXR¹²);
R¹², R^{12′}, and R^{12″} represent each independently a hydrogen atom, an alkyl group optionally having a substituent group, an aryl group optionally having a substituent group, or a heteroaryl group optionally having a substituent group; and
m represents an integer from 1 to 20,
with the proviso that at least four of R⁷, R⁸, R⁹, R¹⁰ and R¹¹ are not simultaneously a hydrogen atom.
(3) A compound represented by the following formula (III) or a salt thereof, or a hydrate thereof,

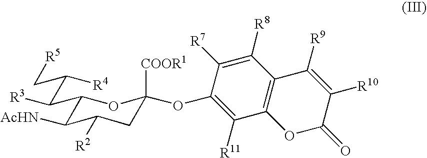

wherein:
R¹, R², R³, R⁴, and R⁵ are the same as defined in (1); and
R⁷, R⁸, R⁹, R¹⁰, R¹¹ are the same as defined in (2).
(4) A compound represented by the following formula (IV) or a salt thereof, or a hydrate thereof,

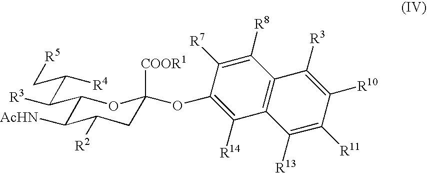

wherein:
R¹, R², R³, R⁴, and R⁵ are the same as defined in (1);
R⁷, R⁸, R⁹, R¹⁰, R¹¹, R¹³, and R¹⁴ represent each independently a hydrogen atom, —NO₂, —F, —Cl, —Br, —I, —N₃, —CN, —Ph, —CX₂R¹², —CHXR¹², —(CH₂)ₘR¹², —CR¹²═CR^{12′}—R^{12″}, —C≡C—R¹², —OP(═O)R¹²(OR^{12′}), —P(═O)R¹²(OR^{12′}), —OPR¹²(OR^{12′}), —PR¹²(OR^{12′}), —OP(R¹²)₂, —P(R¹²)₂, —OS(═O)₂R¹², —S(═O)₂R¹², —OS(═O)R¹², —S(═O)R¹², —C(═O)R¹², or —NR¹²C(═O)R^{12′}; and
R¹², R^{12′}, R^{12″}, and m are the same as defined in (2).
(5) A compound represented by the following formula (V) or a salt thereof, or a hydrate thereof,

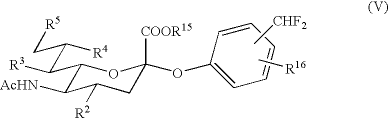

wherein:
R², R³, R⁴ and R⁵ are the same as defined in (1);
R¹⁵ represents a hydrogen atom or an alkyl group; and
R¹⁶ represents an alkyl group optionally having a substituent group, an alkenyl group optionally having a substituent group, an alkynyl group optionally having a substituent group, an aryl group optionally having a substituent group, a heteroaryl group optionally having a substituent group, —NO₂, —N₃, an alkyloxy group, an aminooxy group, an alkyloxycarbonyl group, —NHCOR¹⁷ (wherein R¹⁷ represents a hydrogen atom, an alkyl group optionally having a substituent group, an alkenyl group optionally having a substituent group, an alkynyl group optionally having a substituent group, an aryl group optionally having a substituent group, a heteroaryl group optionally having a substituent group, a carboxyl group, an alkyloxycarbonyl group, an amide group, or a marker group), or a marker group.
(6) A compound represented by the following formula (VI) or a salt thereof, or a hydrate thereof,

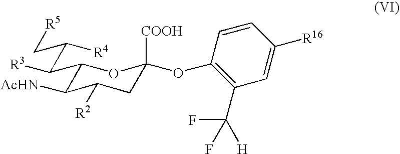

wherein:
R¹, R², R³, R⁴ and R⁵ are the same as defined in (1); and
R¹⁶ is the same as defined in (5).
(7) The compound or a salt thereof, or a hydrate thereof described in (6), wherein R¹⁶ of the formula (VI) is represented by the following formula,

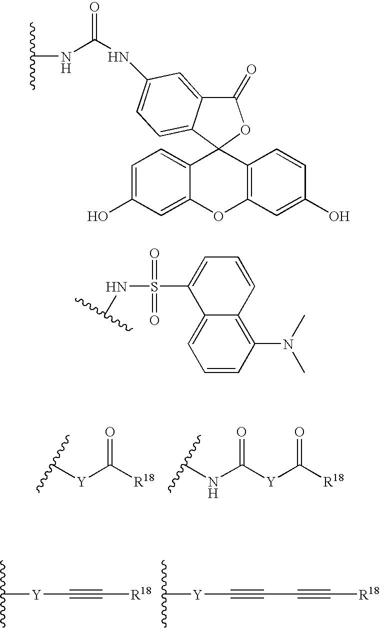

wherein:
Y is selected from the group consisting of —NHCO—, —S—, —O—, —NH—, a C₁₋₂₀ alkylene group, a C₁₋₂₀ arylene group, and a marker group; and
R¹⁸ represents a hydrogen atom, an alkyl group optionally having a substituent group, an alkenyl group optionally having a substituent group, an alkynyl group optionally having a substituent group, an aryl group optionally having a substituent group, a heteroaryl group optionally having a substituent group, —NO₂, an amino group optionally having a substituent group, —N₃, a hydroxyl group, an alkyloxy group, an aminooxy group, an alkyloxycarbonyl group, —NHCOR¹⁷ (wherein R¹⁷ is the same as defined in (5)), or a marker group.
(8) The compound or a salt thereof, or a hydrate thereof described in any of (1) to (7), wherein R¹ represents a hydrogen atom or alkyl; R² to R⁵ represent each independently a hydroxyl group, an acyloxy group, an amino group, or a guanidyl group.
(9) The compound or a salt thereof, or a hydrate thereof described in any of (1) to (8), wherein R² is an amino group or a guanidyl group.
(10) A compound obtained by reacting the compound described in any of the above (1) to (9) with a carrier containing a functional group selected from the group consisting of an optionally protected aminooxy group, N-alkylaminooxy group, hydrazide group, azide group, thiosemicarbazide group, 1,2-dithiol group, alkyl iodide group, acid anhydride, acid fluoride, acid chloride, acid bromide, tetrafluorophenyl ether group, N-hydroxysuccinimide ether group, haloalkyl group, haloacyl group, amino group, hydroxyl group, aldehyde group, acetylene group, ketone group, carboxyl group, and cysteine residue.
(11) The compound described in the above (10), which is selected from the group consisting of polymers or copolymers of:
a) acrylamides, methacrylamides, acrylic acids, methacrylic acids, styrenes, or fatty acid vinylesters;
b) silica carriers, resin carriers, magnetic beads, or metal carriers;
c) compounds represented by the following formulas:

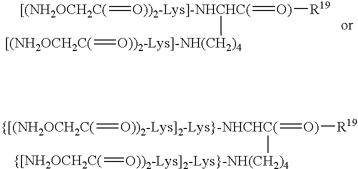

[(NH₂OCH₂C(═O))₂—Lys]—NHCHC(═O)—R¹⁹[(NH₂OCH₂C(═O))₂—Lys]—NH(CH₂)₄

or
([(NH₂OCH₂C(═O))₂—Lys]₂—Lys]—NHCHC(═O)—R¹⁹([(NH₂OCH₂C(═O))₂—Lys]₂—Lys)—NH(CH₂¹)₄
   wherein R¹⁹ represents a hydroxyl group or an amino group, Lys represents lysine, and Cys represents cysteine,

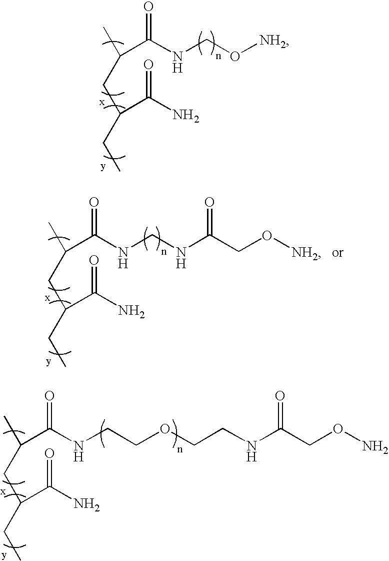

wherein n represents an integer from 1 to 15, and x:y is 1:0 to 1:1000; and
d) compounds represented by the following formula,

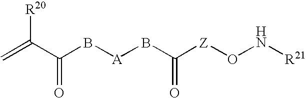

wherein A represents a C₂₋₁₂ alkylene group optionally having a substituent group, B represents —NH— or —O—, R²⁰ represents a hydrogen atom or a methyl group, Z represents a C₁₋₅ alkylene group optionally having a substituent group, and R²¹ represents a hydrogen atom or a protecting group of amino group.
(12) The compound described in any of the above (1) to (11), has covalent bond with a physiologically active substance.
(13) The compound described in any of the above (1) to (12), has covalent bond or coordinate bond with silver.
(14) The compound described in any of the above (1) to (13), has covalent bond or coordinate bond with a magnetic substance.
(15) A drug comprises the compound described in any of the above (1) to (14).
(16) A therapeutic agent for an infectious disease comprises the compound described in any of the above (1) to (14).
(17) A therapeutic agent for an infectious disease involving neuraminidase comprises the compound described in any of the above (1) to (14).
(18) An agent for detecting neuraminidase-producing microorganisms comprises the compound described in any of the above (1) to (14).
(19) An agent for detecting a neuraminidase-expressing site comprises the compound described in any of the above (1) to (14).
(20) An agent for isolating and detecting neuraminidase comprises the compound described in any of the above (1) to (14).
(21) A neuraminidase inhibitor comprising the compound described in any of the above (1) to (14).
(22) A neuraminidase analysis method comprising the steps of: adding the compound described in any of the above (1) to (14) to a solution containing neuraminidase to capture the neuraminidase; adding protease to the solution to digest the neuraminidase into peptide fragments; and then analyzing the peptide fragments by mass spectrometry.
(23) A protein analysis method comprising the steps of: adding the compound described in any of the above (1) to (14) to a solution containing neuraminidase to capture the neuraminidase; and analyzing proteins bonded to the neuraminidase by ELISA method, fluorescence or staining method.
(24) A disease diagnostic method which comprises the steps of: adding the compound described in any of the above (1) to (14) to a solution or suspension containing biological specimens, bacteria and/or viruses; capturing biological specimens, bacteria and/or viruses containing neuraminidase; then performing antigenic analysis for the captured biological specimens, bacteria and/or viruses using antibodies or analyzing DNA or RNA using PCR.

The present invention provides a novel compound having an inhibitory activity against neuraminidase through covalent bond formation. The compound of the present invention is useful as an active ingredient for diagnostic agents and therapeutic agents for diseases involving neuraminidase. Further, neuraminidase can easily be detected using a compound of Formula (I) having a chromophore, a fluorescent group, or the like bonded thereto. Furthermore, by using a compound of Formula (I) having a physiologically active substance bonded thereto, the physiologically active substance can effectively be delivered to a target site.

This specification includes the contents disclosed in the specification and/or drawings of Japanese Patent Application No. 2004-365660, which is a priority document of the present application.

### BRIEF DESCRIPTION OF THE DRAWINGS

FIG. 1 is a diagram showing hydrolysis amounts at a glycoside bond site of p-nitrophenyl-α-N-acetyl neuraminic acid of neuraminidase after neuraminidase produced by Vibro-Choralie and a compound 6 are pre-incubated for 10 minutes under the conditions shown in Example 13 (the original enzyme activity is represented as 1 relatively.);

FIG. 2 is a diagram showing a correlation between the amount of inhibitor (concentration) and hydrolysis rate of neuraminidase substrate when neuraminidase produced by Vibro-Choralie is processed with the compound 6 under the conditions shown in Example 13;

FIG. 3 is a diagram showing an antilogarithmic plot of FIG. 2 and an inhibition constant of the compound 6 against neuraminidase produced by Vibro-Choralie calculated thereby;

FIG. 4 shows MALDI TOF-MS spectra before (upper) and after (lower) processing with an anti-Dansyl antibody column on protease degradation products of neuraminidase processed by an inhibitor of the present invention; and

FIG. 5 shows MALDI TOF-MS spectrum of protease-digested fragment after neuraminidase produced by Vibro-Choralie is captured by using a resin 12 coated with a neuraminidase inhibitor of the present invention and washed.

### BEST MODE FOR CARRYING OUT THE INVENTION

A compound of the present invention is represented by the following formula (I),

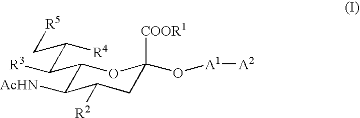

wherein
A¹ represents an aryl group optionally having a substituent group or a heteroaryl group optionally having a substituent group;
A² represents —CX₂R⁶ or —CHXR⁶ wherein X represents —F, —Cl, Br, or —I;
R¹ represents a hydrogen atom or an alkyl group optionally having a substituent group;
R², R³, R⁴, and R⁵ represent each independently —OC(═O)R⁶, —OR⁶, —N(R⁶)₂, —N₃, —NHC(═NH)NHR⁶, —NHCOR⁶, —OSO₃R⁶, —OPO₃(R⁶)₂, F, Cl, Br, or I; and
R⁶ represents each independently a hydrogen atom, an alkyl group optionally having a substituent group, an aryl group optionally having a substituent group, or an optionally substituted heteroaryl group.

Examples of aryl groups used herein include aromatic hydrocarbons having 1 to 30 carbon atoms (preferably 1 to 15) such as phenyl, phenylene, benzyl, benzylidene, pyrenyl, biphenyl, biphenylene, naphthyl, naphthylene, naphthalenyl, fluorenyl, and anthracenyl groups.

Examples of hetero atoms mentioned herein include nitrogen atom, oxygen atom, sulfur atom, fluorine atom, chlorine atom, bromine atom, iodine atom, boron atom, phosphorus atom, and silicon atom.

Examples of heteroaryl groups mentioned herein include heteroaryl having in its ring one or more hetero atoms described above, preferably nitrogen atom, oxygen atom, and/or sulfur atom. Examples thereof include coumarinyl, methylumbelliferyl, flavonyl, pyridyl, pyridazinyl, imidazolyl, quinolyl, adenyl, uracil, isoquinolyl, pyrimidyl, pyrrolidyl, indolyl, and indolynyl groups.

Examples of alkyl groups mentioned herein include linear or branched alkyl groups having 1 to 20 carbon atoms (preferably 1 to 6) such as methyl, ethyl, n-propyl, isopropyl, n-butyl, sec-butyl, tert-butyl, pentyl, and hexyl.

Examples of alkenyl groups mentioned herein include linear or branched alkenyl groups having 2 to 20 carbon atoms (preferably 2 to 6) such as ethenyl group, 1-propenyl group, isopropenyl group, 2-butenyl group, 1,3-butadienyl group, 2-pentenyl group, 2-hexenyl group, cyclopropenyl group, cyclopentenyl group, and cyclohexenyl group.

Examples of alkynyl groups mentioned herein include linear or branched alkynyl groups having 2 to 20 carbon atoms (preferably 2 to 6) such as ethynyl group, 1-propynyl group, 2-propynyl group, 1-butynyl group, and 2-butynyl group.

Examples of alkyloxy groups mentioned herein include alkyloxy groups having the above-described linear or branched alkyl group with 1 to 20 carbon atoms (preferably 1 to 6). Specific examples thereof include methoxy, ethoxy, propoxy, butoxy, and pentyloxy.

Examples of amino groups optionally having a substituent group mentioned herein include amino groups mono- or di-substituted by linear or branched alkyl group or acyl group having 1 to 20 carbon atoms (preferably 1 to 6) such as amino, methylamino, dimethylamino, ethylamino, diethylamino, ethylmethyl amino, propylamino, dipropylamino, and acetylamino.

The ether group mentioned herein is a group —R^{a}OR^{b}. Herein, group R^{a} is a linear or branched alkylene group having 1 to 20 carbon atoms (preferably 1 to 6) such as methylene, ethylene, propylene, and isopropylene, and group R^{b} is a linear or branched alkyl group having 1 to 20 carbon atoms (preferably 1 to 6) such as methyl, ethyl, n-propyl, isopropyl, n-butyl, sec-butyl, tert-butyl, pentyl and hexyl.

The ester group mentioned herein is a group —COOR^{c}. Herein, group R^{c} is a liner or branched alkyl group having 1 to 20 carbon atoms (preferably 1 to 6) such as methyl, ethyl, n-propyl, isopropyl, n-butyl, sec-butyl, tert-butyl, pentyl and hexyl.

The acyl group mentioned herein is alkyl carbonyl or aryl carbonyl. Alkyl carbonyl and aryl carbonyl are optionally substituted by the substituent groups mentioned for “alkyl optionally having a substituent group” or “aryl optionally having a substituent group”, preferably acetyl group.

In this description, examples of substituent groups in “alkyl optionally having a substituent group”, “alkenyl optionally having a substituent group”, “alkynyl optionally having a substituent group”, “aryl optionally having a substituent group”, and “heteroaryl optionally having a substituent group” include —OH, —NHNH₂, —NHNHR, —N(R)₂, —NHC(═NH)NHR, —ONH₂, —NO₂, —CN, —OC(═O)R, —OR, —ONHR, —N₃, —CHO, —C(═O)R, —NHCOR, —OSO₃R, —OPO₃(R)₂, —F, —Cl, —Br, —I or marker groups (wherein R is alkyl, acyl, aryl, heteroaryl or the like).

In A¹, R¹², R^{12′}, R^{12″}, R¹⁶, and R¹⁸, examples of substituent groups in “alkyl optionally having a substituent group”, “alkenyl optionally having a substituent group”, “alkynyl optionally having a substituent group”, “aryl optionally having a substituent group”, and “heteroaryl optionally having a substituent group” include —ONH₂, —NO₂, —CN, —OC(═O)R, —OR, —ONHR, —N₃, —CHO, —C(═O)R, —NHCOR, —OSO₃R, —OPO₃(R)₂, —F, —Cl, —Br, —I or marker groups (wherein R is alkyl, acyl, aryl, heteroaryl or the like).

The marker group mentioned herein is, for example, a physically, electromagnetically, optically, or chemically detectable group. Examples thereof include groups, which are well known in the field of diagnosis and detection for medical and biochemical research, such as chromophores, fluorescent groups, soluble polymers, polymer fine particles, metal fine particles, magnetic fine particles, stable free radicals, radioactive isotopes, enzymes, lectins, antibodies, peptides and oligonucleotides, and groups including those.

In this description, the chromophore is a functional group that absorbs an electromagnetic wave of UV-VIS (near-ultraviolet to visible) range (preferably 200 to 800 nm), and the fluorescent group is a functional group that emits an electromagnetic wave of VIS range (preferably 300 to 900 nm) by absorbing an electromagnetic wave of UV-VIS range. Examples of those chromophores and fluorescent groups include fluorescein group, dansyl group, AMCA group, DIDS group, pyridylamino group, rhodamine group, tetramethylrhodamine group, coumarin group, 7-methoxycoumarin group, pyrene group, NBD group, and Q-dot.

Preferable examples are represented by the following formulas.

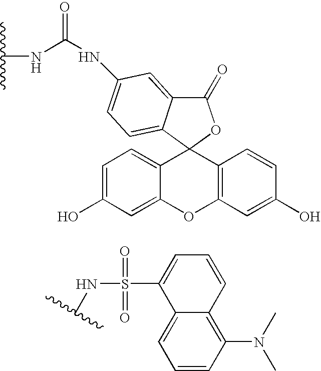

Specifically, when A¹ of the formula (I) is a phenyl group and a derivative thereof, the compound of the present invention is represented by the following formula,

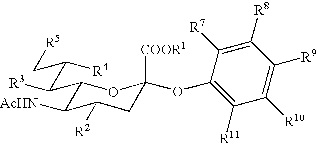

wherein:
R¹, R², R³, R⁴, and R⁵ are the same as defined in (1);
R⁷, R⁸, R⁹, R¹⁰, and R¹¹ represent each independently a hydrogen atom, —NO₂, —F, —Cl, —Br, —I, —N₃, —CN, —Ph, —CX₂R¹², —CHXR¹², —(CH₂)ₘR¹², —CR¹²═CR^{12′}—R^{12″}, —C≡C—R¹², —OP(═O)R¹²(OR^{12′}), —P(═O)R¹²(OR^{12′}), —OPR¹²(OR ^{12′}), —PR¹²(OR^{12′}), —OP(R¹²)₂, —P(R¹²)₂, —OS(═O)₂R¹², —S(═O)₂R¹², —OS(═O)R¹², —S(═O)R¹², —C(═O)R¹², or —NR¹²C(═O)R^{12′} (wherein at least one of R⁷, R⁹, and R¹¹ represents each independently —CX₂R¹² or —CHXR¹²);
R¹², R^{12′}, and R^{12″} represent each independently a hydrogen atom, an alkyl group optionally having a substituent group, an aryl group optionally having a substituent group, or a heteroaryl group optionally having a substituent group; and
m represents an integer from 1 to 20,
with the proviso that at least four of R⁷, R⁸, R⁹, R¹⁰ and R¹¹ are not simultaneously a hydrogen atom.

Further, when A¹ of the formula (I) is coumarinyl group and a derivative thereof, the compound of the present invention is represented by the following formula,

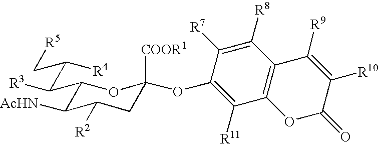

wherein:
R¹, R², R³, R⁴, and R⁵ are the same as defined in (1); and
R⁷, R⁸, R⁹, R¹⁰, and R¹¹ are the same as defined in (2).

Furthermore, when A¹ of the formula (I) is a naphthyl group and a derivative thereof, the compound of the present invention is represented by the following formula,

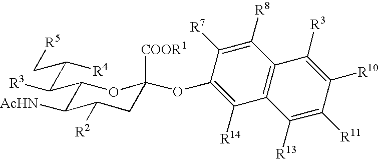

wherein:
R¹, R², R³, R⁴, and R⁵ are the same as defined in (1);
R⁷, R⁸, R⁹, R¹⁰, and R¹¹, R¹³ and R¹⁴ represent each independently a hydrogen atom, —NO₂, —F, —Cl, —Br, —I, —N₃, —CN, —Ph, —CX₂R¹², —CHXR¹², —(CH₂)ₘR¹², —CR¹²═CR^{12′}—R^{12″}, —C≡C—R¹², —OP(═O)R¹²(OR^{12′}), —P(═O)R¹²(OR^{12′}), —OPR¹²(OR^{12′}), —PR¹²(OR^{12′}), —OP(R¹²)₂, —P(R¹²)_{2, —OS(═O)}₂R¹², —S(═O)₂R¹², —OS(═O)R¹², —S(═O)R¹², —C(═O)R¹², or —NR¹²C(═O)R^{12′}; and
R¹², R^{12′}, R^{12″}, and m are the same as defined in (2).

Further, when A¹ and A² of the formula (I) are a phenyl group and a difluoromethyl group, respectively, the compound of the present invention is represented by the following formula,

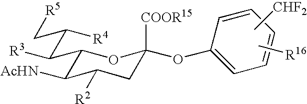

wherein:
R², R³, R⁴ and R⁵ are the same as defined in (1);
R¹⁵ represents a hydrogen atom or an alkyl group; and
R¹⁶ represents an alkyl group optionally having a substituent group, an alkenyl group optionally having a substituent group, an alkynyl group optionally having a substituent group, an aryl group optionally having a substituent group, a heteroaryl group optionally having a substituent group, —NO₂, —N₃, an alkyloxy group, an aminooxy group, an alkyloxycarbonyl group, —NHCOR¹⁷ (wherein R¹⁷ represents a hydrogen atom, an alkyl group optionally having a substituent group, an alkenyl group optionally having a substituent group, an alkynyl group optionally having a substituent group, an aryl group optionally having a substituent group, a heteroaryl group optionally having a substituent group, a carboxyl group, an alkyloxycarbonyl group, an amide group, or a marker group), or a marker group.

In addition, when A¹ of the formula (I) is a phenyl group and A² thereof is a difluoromethyl group that is adjacent to sialic acid-binding site and has a further substituent group introduced to a p-site through the sialic acid-binding site, the compound of the present invention is represented by the following formula,

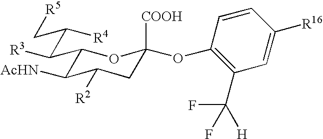

wherein:
R¹, R², R³, R⁴ and R⁵ are the same as defined in (1); and
R¹⁶ is the same as defined in (5).

Further, the substituent group R¹⁶ is preferably one represented by the following formulas,

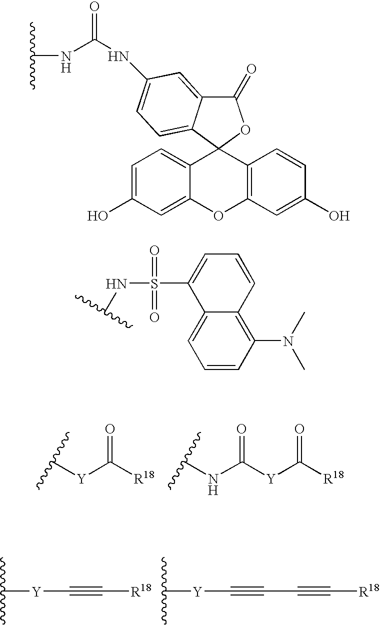

wherein:
Y is selected from the group consisting of —NHCO—, —S—, —O—, —NH—, C₁₋₂₀ alkylene group, C-₁₋₂₀ arylene group, and a marker group;
R¹⁸ represents a hydrogen atom, an alkyl group optionally having a substituent group, an alkenyl group optionally having a substituent group, an alkynyl group optionally having a substituent group, an aryl group optionally having a substituent group, a heteroaryl group optionally having a substituent group, —NO₂, an amino group optionally having a substituent group, —N₃, a hydroxyl group, an alkyloxy group, an aminooxy group, an alkyloxycarbonyl group, —NHCOR¹⁷ (wherein R¹⁷ is the same as defined in (5)), or a marker group.

The compound of the present invention may be bonded to various carriers. Such binding may be formed directly to A² or A³ of the formula (I) of the present invention or via a spacer or the like. Preferably, used are carriers containing the following functional groups: [the functional group selected from the group consisting of an optionally protected aminooxy group, N-alkylaminooxy group, hydrazide group, azide group, thiosemicarbazide group, 1,2-dithiol group, alkyl iodide group, acid anhydride, acid fluoride, acid chloride, acid bromide, tetrafluorophenyl ether group, N-hydroxysuccinimide ether group, haloalkyl group, haloacyl group, amino group, hydroxyl group, aldehyde group, acetylene group, ketone group, carboxyl group, cysteine residue, and metal surface.

Still further, the carrier to be bonded to the compound of the present invention is preferably selected from the group consisting of polymers or copolymers of:
a) acrylamides, methacrylamides, acrylic acids, methacrylic acids, styrenes, or fatty acid vinylesters;
b) silica carriers, resin carriers, magnetic fine particles, or metal carriers;
c) compounds represented by the following formulas:

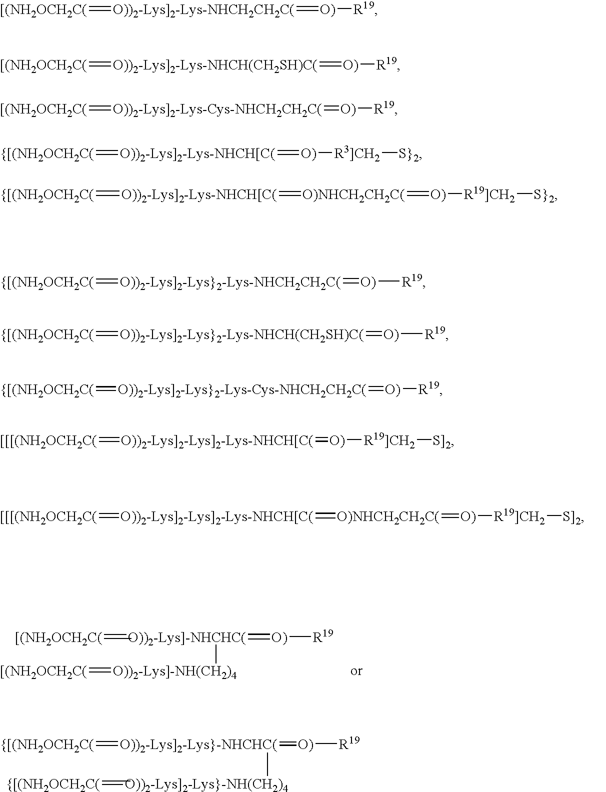

wherein R¹⁹ represents a hydroxyl group or an amino group, Lys represents lysine, and Cys represents cysteine,

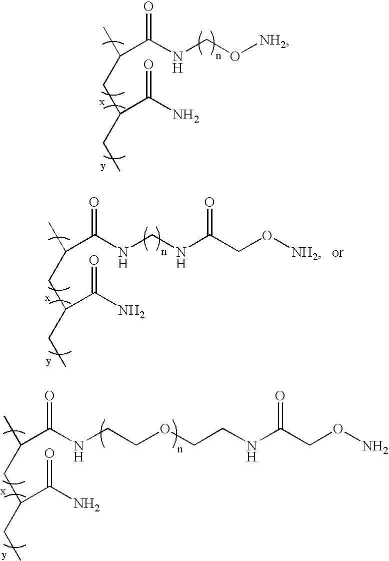

wherein n represents an integer from 1 to 15, and x:y is 1:0 to 1:1000; and
d) compounds represented by the following formula,

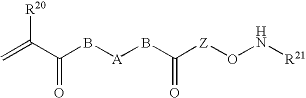

wherein A represents a C₂₋₁₂ alkylene group optionally having a substituent group, B represents —NH— or —O—, R²⁰ represents a hydrogen atom or a methyl group, Z represents a C₁₋₅ alkylene group optionally having a substituent group, and R²¹ represents a hydrogen atom or a protecting group of amino group.

The shape of the carrier of the present invention is not limited, and the carrier may have shape such as particle, chip, film, fiber, monolith, and gel. As a component of the carrier, those commonly used in this technical field can be used, and the component is not particularly limited. Both of organic and inorganic materials are usable. Examples of the organic material include: fluorine resins such as polytetrafluoroethylene (PTFE) and polyvinylidene fluoride (PVDF); polyolefins such as polypropylene, polyethylene, polybutene, polystyrene, polyvinyl chloride, polyvinyl alcohol, and polyvinyl acetate; polyamides such as nylon (for example, Nylon 6, Nylon 66, Nylon 11, Nylon 12, and Nylon MXD6); polyesters such as polybutylene terephthalate, polyethylene terephthalate, and polytrimethylene terephthalate; and polymer materials such as polycarbonate, polyurethane, polylactic acid, polyimide resin, ABS resin (Acrylonitrile Butadiene Styrene resin), acrylic resin, methyl pentene resin, phenol resin, melamine resin and epoxy resin. Examples of the inorganic materials include: metals such as gold, silver, copper, iron, aluminum, tungsten, cobalt, molybdenum, chromium, platinum, titanium, and nickel; alloys such as stainless, hastelloy, inconel, monel, and duralumin; metal oxides such as magnetite and ferrite; laminated products of the above-mentioned metal and ceramics; silicon; silica; glass; fused quartz; synthetic quartz; alumina; sapphire; ceramics; forsterite; and photosensitive glass.

The compound of the present invention may be bonded to a metal fine particle. The metal fine particle is a fine particle composed of a single metal or a metal complex. Exemplary components of the fine particle include gold, silver, copper, iron, zinc, selenium, cadmium, cobalt, and nickel. The component may be a metal complex or a complex of metal and nonmetal, and an oxide. Representative metal fine particles used in the present invention include fine particles composed of silver alone, magnetic particles composed of, for example, iron, nickel, cobalt, or iron oxide (Fe₃O₄, magnetite), or metal complex fine particles wherein surfaces of magnetic particles are coated with silver. These metal fine particles may be bonded directly to functional groups of aryl groups of the compound represented by formula (I) of the present invention, or via spacers or the like. The bond formation between the metal fine particle surface, and the compound of formula (I) of the present invention or the spacer may be any bond formation such as covalent bond, coordinate bond, physical adsorption, and bond to a coating component by polymer coating. These do not restrict the patent for the present invention, but covalent bond to sulfur atom (thiol or disulfide) is preferred since simple and relatively strong bond formation is expected. Further, the functional group of a linker at a side bonded to the compound of formula (I) of the present invention is preferably: [a functional group selected from the group consisting of an optionally protected aminooxy group, an N-alkylaminooxy group, a hydrazide group, an azide group, a thiosemicarbazide group, a 1,2-dithiol group, an alkyl iodide group, an ester group, an acid anhydride, an acid fluoride, an acid chloride, an acid bromide, a tetrafluorophenyl ester group, an N-hydroxysuccinimide ester group, a haloalkyl group, a haloacyl group, an amino group, a hydroxyl group, an aldehyde group, an acetylene group, a ketone group, a carboxyl group, and a cysteine residue].

The metal fine particle mentioned herein has an average particle diameter of 1 nm to 5 μm, preferably 10 nm to 1 μm, and preferably usable for rapid isolation by filtration, ultrafiltration, centrifugation, or a magnet using magnetism.

As a nonmagnetic metal component, silver is preferable due to its small influence on human body and antimicrobial activity. Silver alone or a complex thereof may be used. The complex may be an alloy compound containing a silver component or a complex in which a particle nuclear component composed of a material other than silver is coated with silver. As the particle nuclear component, those commonly used in the technical field can be used without particular limitation, and both of inorganic and organic materials can be used.

The magnetic fine particle mentioned herein preferably contains a ferromagnetic substance. Examples of the ferromagnetic substances include iron, cobalt, nickel, gadolinium, terbium, dysprosium, holmium, erbium and thulium, and alloys containing these (for example, iron-cobalt alloy), and oxides and complex oxides [for example, magnetite (Fe₃O₄), maghemite (γ-Fe₂O₃), manganese zinc ferrite (Mn₁₋ₓZnₓFe₂O₄), and barium ferrite (BaFe₁₂O₁₉) particles].

The compound of the present invention may be bonded to a physiologically active substance. The physiologically active substance is a substance that gives some characteristic influence on biological physiology or behavior. Examples thereof include: proteins and peptides (for example, enzymes such as human serum albumin, fibrinogen, urokinase, or streptokinase, and antigenic epitope peptide); hormones (for example, adrenocorticotropic hormone and thyroid stimulating hormone); immune antibodies (for example, IgG and fragments thereof F(ab′)₂, Fab′, and Fab); antibiotics (for example, penicillin, erythromycin, and vibramycin); anticancer agents (for example, bleomycin, gefitinib, cisplatin, and mitomycin); biotins, neurotransmitters, saccharides, fatty acids, amino acids, and drugs. These physiologically active substances may be directly bonded to the compound of formula (I) of the present invention so that the functional group of the substance is bonded to the functional group of aryl group of the compound, or bonded thereto via a spacer or the like.

The compound of the present invention is produced, for example, according to the following method.

When sialic acid is used as a starting material, carboxyl group of sialic acid is converted to ester. For example, conversion to ester is conducted as follows. Next, using halogenated acyl, for example, a hydroxyl group is converted to an acyl group (preferably acetyl) and a hydroxyl group at the anomeric position is converted to a halogen (preferably chloro).

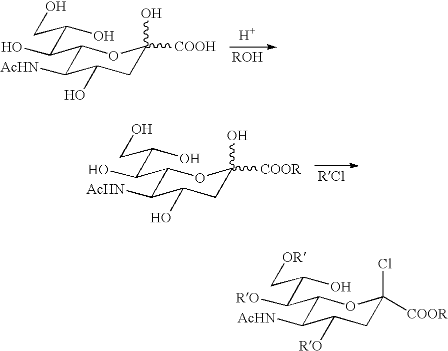

Next, glycosylation reaction is conducted using a phenol derivative having an aldehyde group, an α-keto alkyl group, a hydroxymethylene group, or an α-hydroxyalkyl group as a sugar receptor. Finally, an aldehyde group is fluorinated using a fluorinating agent such as Et₂NSF₃, providing the compound of the present invention.

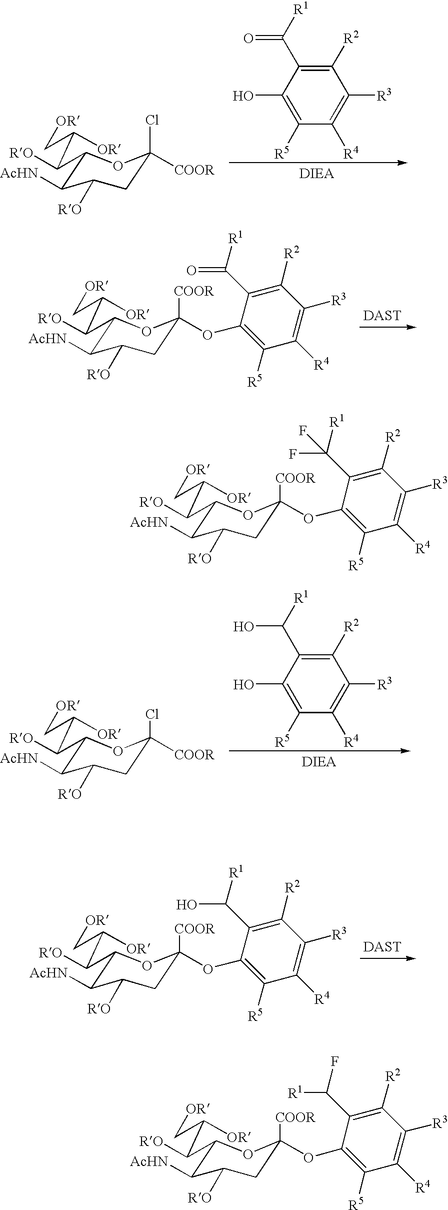

Salts of the compound of the present invention preferably are pharmaceutically acceptable, and examples thereof include those derived from inorganic or organic acids or bases. Specific examples include hydrochloride, hydrobromic acid salt, sulfate, nitrate, perchlorate, fumarate, maleate, phosphate, glycolate, lactate, salicylate, succinic acid salt, toluene-p-sulfonate, tartrate, acetate, citrate, methanesulfonate, formate, benzoate, malonate, naphthalene-2-sulfonate, trifluoroacetate, benzenesulphonate, amine salt and ammonium salt.

The compound of the present invention inhibits enzyme activity of neuraminidase. The compound of the present invention exhibits not competitive inhibitory action but irreversible inhibitory action against neuraminidase.

The compound of the present invention cleaves glycoside bond and detaches aglycone near an active site of neuraminidase. This aglycone site is electrophilic, and thus is reacted with and bonded to a nucleophilic functional group near the active site of neuraminidase, thereby inhibiting enzyme activity of neuraminidase. Further, since this bond is a covalent bond, the inhibitory activity is irreversible.

The compound of the present invention exhibits neuraminidase inhibitory activity, and thus useful as an active ingredient of a drug such as therapeutic agents, preventive agents, and diagnostic agents for infectious diseases such as neuraminidase-possessing microorganisms (viruses and bacteria, for example, bactera such as Vibrio cholerae, Clostridium perfringe, gas bacillus, Streptococcus pneumoniae, Helicobacter pylori, and Arthrobacter sialophilus; parasites such as Trypanosoma cruzi, Trypanosoma brucei, Cryptosporidium, and Cryptosporidium parvum; and viruses such as influenza virus, parainfluenza virus, fowl plague virus, mumps virus, Sendai virus, and Newcastle disease virus).

The drug containing the compound of the present invention as an active ingredient is formulated in a proper dosage form in accordance with administration route.

Specific examples of oral formulations include tablet, capsule, powder, granule, and syrup. Specific examples of parenteral formulations include injection, suppository, tape, and ointment. These various formulations can be produced by ordinary methods using an excipient, a disintegrating agent, a binder, a lubricant, a coloring agent, and a diluent, which are commonly used.

Examples of the excipients include lactose, glucose, corn starch, sorbit, and crystalline cellulose. Examples of the disintegrating agents include starch, sodium alginate, gelatin powder, calcium carbonate, calcium citrate, and dextrin. Examples of the binders include dimethyl cellulose, polyvinyl alcohol, polyvinyl ether, methylcellulose, ethyl cellulose, arabic gum, gelatin, hydroxypropylcellulose, and polyvinylpyrrolidone. Examples of the lubricants include talc, magnesium stearate, polyethylene glycol, and hydrogenated vegetable oil.

Further, the above injection may be produced, if necessary, by adding a buffer, a pH adjuster, a stabilizing agent, a tonicity agent, and a preservative.

The content of the compound of the present invention in a pharmaceutical composition of the present invention may vary depending on the dosage form, but the content thereof is usually 0.01 to 50% by weight, preferably 0.1 to 20% by weight with respect to the whole composition.

The dosage may be determined depending on individual condition, considering patients' ages, weights, sexes, difference in disease, and symptom severity. For example, the dosage is 0.01 to 100 mg/kg, preferably 0.1 to 50 mg/kg, which is administered once a day or divided into some dosage units.

A compound using a marker group as a substituent group R^{X} is useful as an active ingredient of a detection agent of neuraminidase. A sample that may contain neuraminidase is contacted with a labeled compound of the present invention, the mixture is observed using a method capable of detecting the marker group, and thereby the presence, concentration, or the like of neuraminidase can be determined. Further, use of the marker group as a probe is effective as methods for isolating neuraminidase and for probing an active site of neuraminidase. Examples of these methods include: a method for identifying neuraminidase in an electrophoresis gel of a reaction solution by using a fluorescent group; a method for identifying neuraminidase by using an antibody recognizing the marker group; and a method for rapidly isolating labeled neuraminidase by using a column having immobilized antibodies recognizing the marker group. Further, the active site of neuraminidase can be predicted by digesting neuraminidase with protease and then isolating peptide fragments having labeling sites.

Further, a compound of the present invention bonded to a carrier is bonded to neuraminidase, and thereafter particles can rapidly be isolated by filtration, ultrafiltration, or centrifugation. Particulate neuraminidase is digested with protease, the produced peptide fragments are measured by a mass spectrometer, and thereby it is possible to conduct the MASCOT calculation of peptide fragment mass pattern and the identification of neuraminidase sequence by MS/MS processing of peptide fragment peaks. This method enables rapid diagnosis and treatment such as infectious disease diagnosis through rapid identification of ex vivo-derived neuraminidase.

Furthermore, the above carrier having the compound of the present invention bonded thereto is contacted to a solution containing neuraminidase to capture the neuraminidase. Protein bonded to the captured neuraminidase is analyzed by ELISA method, fluorescent or staining method, and thereby the protein can be analyzed.

Moreover, the above carrier having the compound of the present invention bonded thereto is contacted to biological specimens (all substances constituting a living body, for example, body fluid such as saliva secreted from living body, blood, cells), and a solution or suspension containing bacteria and/or virus so that biological specimens, and bacteria and/or virus having neuraminidase are captured and isolated. Then, the surfaces of the captured cells are analyzed by antigenic analysis using antibodies, or intracellular DNAs or RNAs are analyzed using PCR, thereby enabling diagnoses on various diseases.

Further, when the compound bonded to silver-containing fine particles is administered to a patient with an infectious disease, it is possible to treat the patient synergistically with antibacterial and antiviral activities exhibited by silver in addition to therapeutic effect on neuraminidase.

Still further, when the particle component is magnetic, administration thereof into the body enables agglutination of the magnetic particles at a site highly expressing neuraminidase, which can be used for disease diagnosis as an MRI contrast material. Particularly, it is known that cell envelope neuraminidase expression increases as inflammation or oncogenic transformation develops. Thus, the magnetic particles can be used for detecting a infectious disease focus, an inflammatory site and tumor. Further, an alternating magnetic field is applied from the outside of the body so as to enable heating, which can be used as a heating element in thermotherapy on cancer or the like.

Further, when a compound using a physiologically active substance as a substituent group is administered, the physiologically active substance can be selectively delivered to a site secreting neuraminidase, which allows use of the compound of the present invention as a carrier of drug delivery system.

### EXAMPLES

Hereafter, the present invention is described in detail, but not limited thereto.

### Example 1

### Synthesis of Compound 3

A compound 3 was synthesized in accordance with the following scheme.

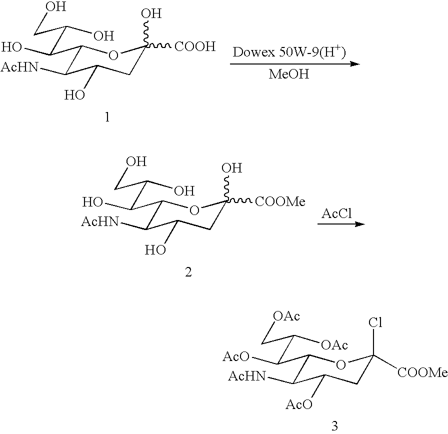

Dowex50W-8 (H⁺ type) (1.0 g) was added to a suspension of sialic acid 1 (1.0 g) in methanol (25 ml), and vigorously shaken for 24 hours. A resin was separated by filtration, and the resultant filtrate was concentrated. Acetyl chloride (5 ml) was added to the obtained residue and vigorously shaken for 24 hours. The reaction solution was concentrated, and toluene azeotropy was conducted three times, followed by vacuum drying, thus providing 1.23 g of compound 3.

### Example 2

### Synthesis of Compound 4

A compound 4 was synthesized in accordance with the following scheme.

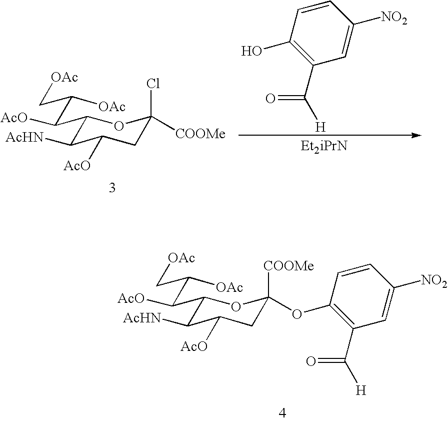

2-hydroxy-5-nitrobenzaldehyde (1.21 g) and diethylisopropylamine (1.69 ml) were sequentially added to a solution of the compound 3 (1.23 g) in acetonitrile (15 ml), and shaken at room temperature for 24 hours. The reaction solution was concentrated, and the obtained residue was purified by silica gel column chromatography [hexane-acetone (4:5)], thereby providing the compound 4 (1.17 g).

### Example 3

### Synthesis of Compound 5

A compound 5 was synthesized in accordance with the following scheme.

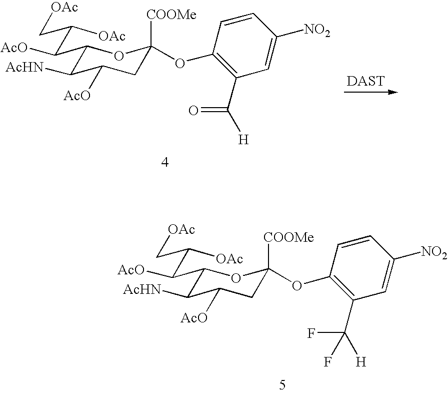

A solution of the compound 4 (1.17 g) in dichloromethane (10 ml) was cooled to −20° C. and DAST (0.51 ml) was dropped to the solution. Thereafter, the reaction solution was heated, while being stirred, to room temperature for one hour. The reaction solution was cooled to 0° C., methanol (1.0 ml) was added thereto, and the mixture solution was concentrated. The obtained residue was purified by silica gel column chromatography [hexane-acetone (4:3)], thereby providing the compound 5 (727 mg, 60%).

### Example 4

### Synthesis of Compound 6

A compound 6 was synthesized in accordance with the following scheme.

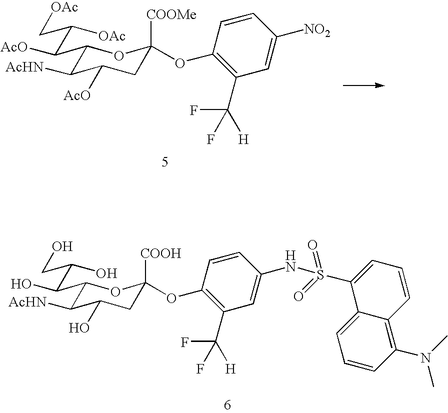

A solution of 0.1 M sodium methoxide in methanol (0.10 ml) was added to a solution of the compound 5 (100 mg) in dried methanol (5 ml), and stirred for one hour. Then, water (2 ml) was added thereto and sodium methoxide was timely added so that the mixture kept a pH of 11 to 12. It was confirmed that the reaction product aggregated at Rf=0.1 by TLC [chloroform-methanol (2:1)]. Thereafter, 1% acetic acid solution was dropped to neutralize the reaction solution. Next, palladium-activated charcoal catalyst was added to the reaction solution, and vigorously stirred for one hour under hydrogen atmosphere. After replacement with nitrogen atmosphere in a reaction vessel, the catalyst was separated by sellite filtration and the obtained filtrate was concentrated. The residue was dissolved in methanol, triethylamine (42 μl) and dansyl chloride (61 mg) were sequentially added thereto, the obtained mixture was stirred at room temperature for 20 hours, and then the reaction solution was concentrated. The obtained residue was purified by silica gel column chromatography [chloroform-methanol-water (10:10:1)], thereby providing the compound 6 (47 mg, 45%).

### Example 5

### Synthesis of Compound 7

A compound 7 was synthesized in accordance with the following scheme.

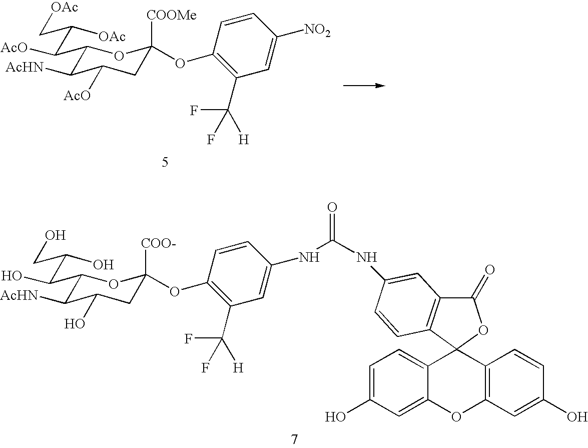

A solution of 0.1 M sodium methoxide in methanol (0.10 ml) was added to a solution of the compound 5 (100 mg) in dried methanol (5 ml), and stirred for one hour. Then, water (2 ml) was added thereto and sodium methoxide was timely added so that the mixture kept a pH of 11 to 12. It was confirmed that the reaction product aggregated at Rf=0.1 by TLC [chloroform-methanol (2:1)]. Thereafter, 1% acetic acid solution was dropped to neutralize the reaction solution. Next, palladium-activated charcoal catalyst was added to the reaction solution, and vigorously stirred for one hour under hydrogen atmosphere. After replacement with nitrogen atmosphere in a reaction vessel, the catalyst was separated by sellite filtration and the obtained filtrate was concentrated. The obtained residue was dissolved in methanol (10 ml), a solution of FITC-isocyanate (100 mg) in acetone (10 was dropped, the mixture was stirred at room temperature for 15 hours, and then the reaction solution was concentrated. The obtained residue was purified by silica gel column chromatography [chloroform-methanol-water (10:10:1)], thereby providing the compound 7 (86 mg, 69%).

### Example 6

### Synthesis of Compound 8

Compound 8 was synthesized in accordance with the following scheme.

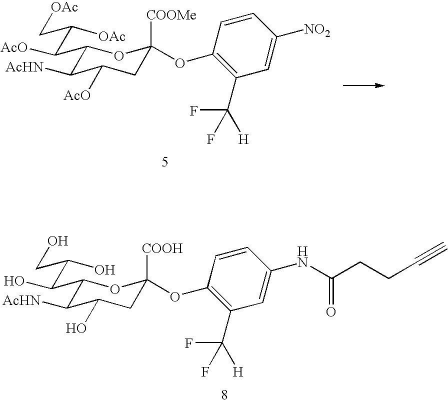

A solution of 0.1 M sodium methoxide in methanol (0.10 ml) was added to a solution of the compound 5 (100 mg) in dried methanol (5 ml), and stirred for one hour. Then, water (2 ml) was added thereto and sodium methoxide was timely added so that the mixture kept a pH of 11 to 12. It was confirmed that the reaction product aggregated at Rf=0.1 by TLC [chloroform-methanol (2:1)]. Thereafter, 1% acetic acid solution was dropped to neutralize the reaction solution. Next, palladium-activated charcoal catalyst was added to the reaction solution, and vigorously stirred for one hour under hydrogen atmosphere. After replacement with nitrogen atmosphere in a reaction vessel, the catalyst was separated by sellite filtration and the obtained filtrate was concentrated. The obtained residue was dissolved in dried methanol (10 ml), 4-pentynoic acid succinimide ester (98 mg) was added in several batches, the mixture was stirred at room temperature for 15 hours, and then the reaction solution was concentrated. The obtained residue was purified by silica gel column chromatography [chloroform-methanol-water (10:10:1)], thereby providing the compound 8 (65 mg, 61%).

This compound 8 can be bonded under mild conditions to various bases (such as solid-phase support), physiologically active substances, marker agents or the like by use of acetylene bond present at the terminal of aglycone.

### Example 7

### Synthesis of Compound 9

A compound 9 was synthesized in accordance with the following scheme.

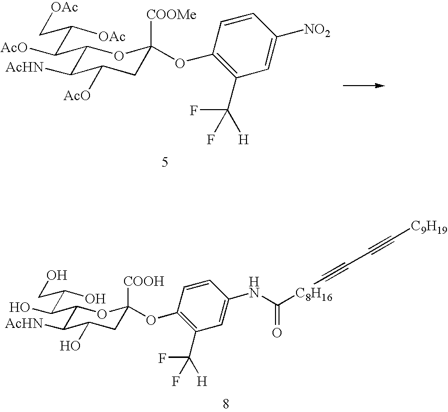

A solution of 0.1 M sodium methoxide in methanol (0.10 ml) was added to a solution of the compound 5 (100 mg) in dried methanol (5 ml), and stirred for one hour. Then, water (2 ml) was added thereto and sodium methoxide was timely added so that the mixture kept a pH of 11 to 12. It was confirmed that the reaction product aggregated at Rf=0.1 by TLC [chloroform-methanol (2:1)]. Thereafter, 1% acetic acid solution was dropped to neutralize the reaction solution. Next, palladium-activated charcoal catalyst was added to the reaction solution, and vigorously stirred for one hour under hydrogen atmosphere. After replacement with nitrogen atmosphere in a reaction vessel, the catalyst was separated by sellite filtration and the obtained filtrate was concentrated. The obtained residue was dissolved in dried methanol (10 ml), 10,12-dodecosadiyonic acid succinimide ester (215 mg) was added in several batches, the mixture was stirred at room temperature for 15 hours, and then the reaction solution was concentrated. The obtained residue was purified by silica gel column chromatography [chloroform-methanol (2:1)], thereby providing the compound 9 (82 mg, 53%).

This compound 9 enables formation of LB film or liposome using fatty chain (alkyl chain) site present at the terminal of aglycone. Further, polymerization of diacetylene structure in the fatty chain by light irradiation or the like enables the formation of a stable single molecular film, liposome or the like. Thus it is possible to produce under mild conditions a clathrate and a copolymer of various bases, physiologically active substances, marker agents or the like.

### Example 8

### Synthesis of Compound 10

A compound 10 was synthesized in accordance with the following scheme.

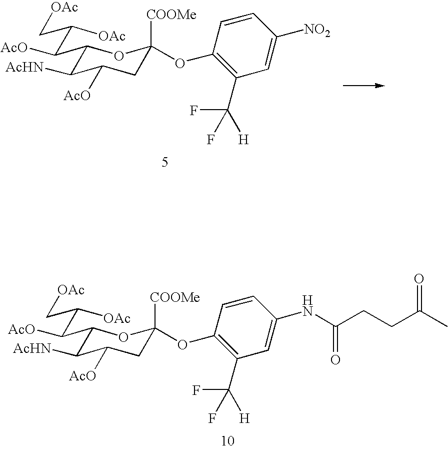

Palladium-activated charcoal catalyst was added to a solution of the compound 5 (300 mg) in ethyl acetate (15 ml), and vigorously stirred under hydrogen atmosphere for 2 hours. After replacement with nitrogen atmosphere in a reaction vessel, the catalyst was separated by sellite filtration and the obtained filtrate was concentrated. The residue was dissolved in dichloromethane (20 ml), levulinic acid (105 mg) and EDC (174 mg) were added, and the obtained solution was stirred overnight. The reaction solution was diluted with chloroform, washed with saturated sodium hydrogen carbonate solution, dried, and concentrated. The obtained residue was purified by silica gel column chromatography [chloroform-methanol (20:1)], thereby providing the compound 10 (331 mg, 105%).

### Example 9

### Synthesis of Compound 11

A compound 11 was synthesized in accordance with the following scheme.

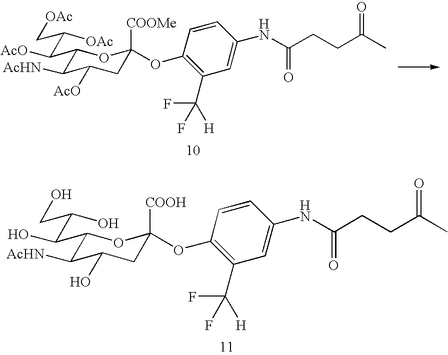

Sodium methoxide (4 mg) was added to a solution of the compound 10 (100 mg) in dried methanol (5 ml), and stirred for 2 hours. The reaction solution was concentrated. 0.1 M sodium hydroxide solution (3 ml) was added to the residue, and stirred for one hour. After neutralization using 5% acetic acid, the reaction solution was concentrated, thereby providing the compound 11 (86 mg, 115%). The compound 11 was used for the next reaction without further purification.

### Example 10

### Preparation of Resin 12 Coated with Neuraminidase Inhibitor

A compound 12 was prepared in accordance with the following scheme.

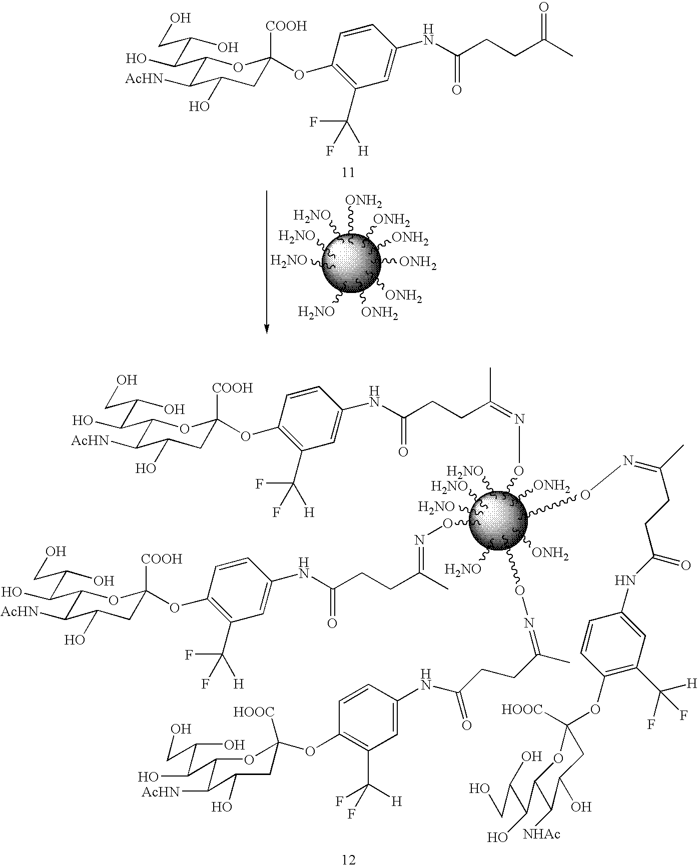

The compound 11 (21 mg) was dissolved in Milli-Q water (5 ml), a suspension of 100 mg of polyacrylamide copolymer (BlotGlyco manufactured by Sumitomo Bakelite Co., Ltd.) having an oxylamino group on its surface in 5 ml of Milli-Q water was added and the mixture was stirred for 2 hours. The reaction solution was separated by filtration, thereby providing a resin 12 coated with neuraminidase inhibitor. The amount of sialic acid on the resin was measured. As a result, the resin had a coating rate of the compound 11 of 81%.

### Example 11

### Preparation of Silver-Coated Magnetic Fine Particle 17 Exhibiting Oxylamino Group

In accordance with the following scheme, an oxylamino group was exhibited on a silver-coated magnetic fine particle.

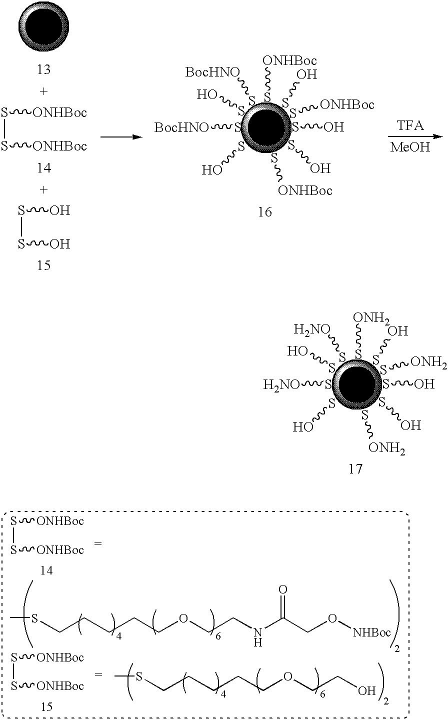

Silver-coated magnetic beads (0.5 mg, average particle diameter of 230 nm manufactured by Hitachi Maxell, Ltd., special order article) were dissolved in 10 mM methanol solution (5 μl) of the compound 14, 10 mM aqueous solution (5 μl) of the compound 15, and Milli-Q water-methanol mixture solution (20 μl, mixture ratio=1:1), followed by stirring overnight. Magnetic beads were collected by using a magnetic, and washed with Milli-Q water and methanol 3 times. After the washing, the magnetic beads were analyzed by MALDI-TOF MS, and as a result, it was confirmed that magnetic particle compound 16 having the compounds 14 and 15 immobilized on the surface thereof was produced. Next, the compound 16 was suspended in 40% TFA/methanol, and the solution was stirred for 2 hours. Using a magnet, the magnetic beads were collected from the reaction solution and washed with methanol and water several times, thereby providing oxylamino compound 17. The production of the compound 17 was confirmed by MALDI-TOF MS.

### Example 12

### Preparation of Silver-Coated Magnetic Fine Particle 18 Exhibiting Neuraminidase Inhibitor

In accordance with the following scheme, a neuraminidase inhibitor of the present invention was exhibited on the surface of a silver-coated magnetic fine particle.

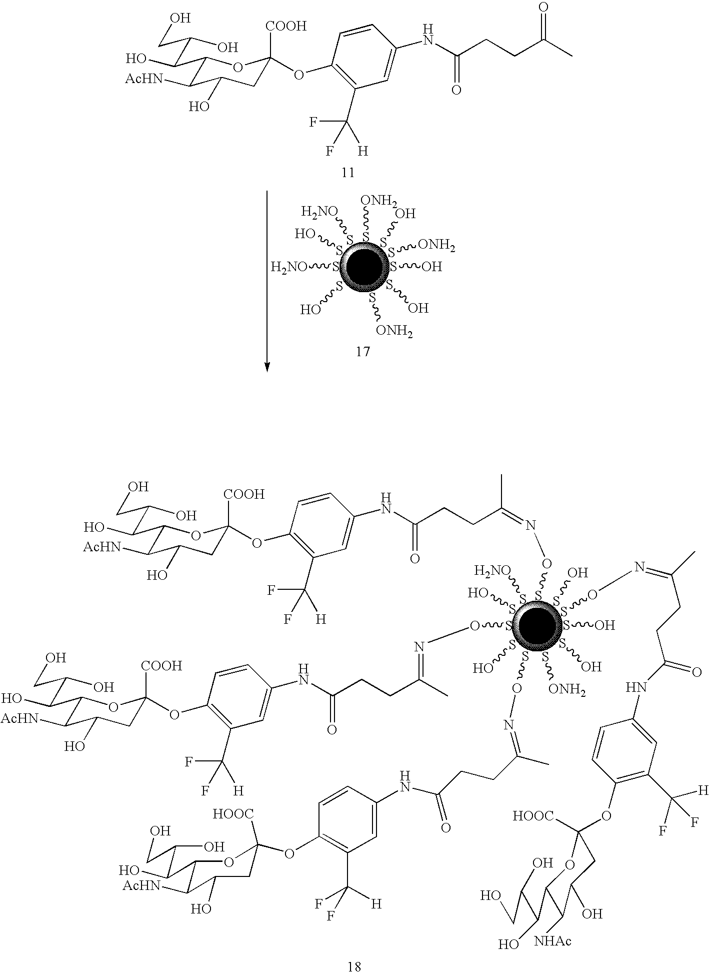

An acetic acid buffer solution (50 μl; pH 5.5) of the compound 11 (50 μg) was added to a suspension of the silver-coated magnetic fine particle 17 exhibiting oxylamino group (0.5 mg) in methanol (10 μl), followed by stirring overnight. Using a magnet, the magnetic beads were collected from the reaction solution, and the magnetic beads were washed with methanol and water several times, thereby providing the silver-coated magnetic bead 18 exhibiting neuraminidase inhibitor. The production of the compound 18 was confirmed by MALDI-TOF MS.

### Example 13

### Examination on Inhibition Activity against Neuraminidase (Derived from Vibrio cholerae) and Determination of Inhibition Constant (Ki and Half-Life of Activity)

Neuraminidase (50 μg) produced by Vibro-Choralie was pre-incubated in 50 mM phosphoric acid-tartaric acid buffer (pH 5.0, 10 μ1) together with the compound 6 (0, 0.05, 0.1, 0.2, and 0.5 mM), after 0, 10 or 30 minutes, 2 μl of the reaction solution was fractionated, respectively. The fractionated and pre-incubated solution was immediately incubated for 5 minutes in 50 mM phosphoric acid-tartaric acid buffer (pH 5.0, 50 μl) using 5 mM p-nitrophenyl-α-N-acetyl neuraminic acid. Then, glycine buffer (pH 11.0, 150 ml) was added to each fractionated solution to stop the reaction. The amount of p-nitrophenol produced by hydrolysis reaction of neuraminidase was calculated by absorbance determination at 405 nm.

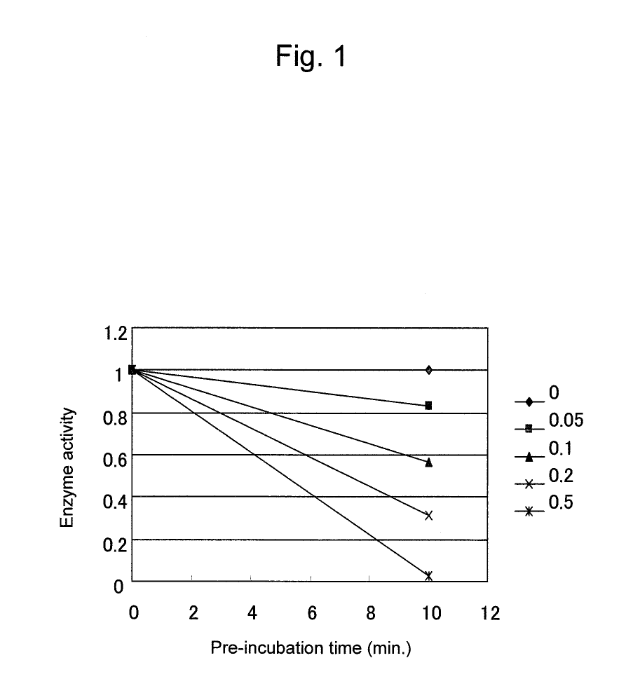

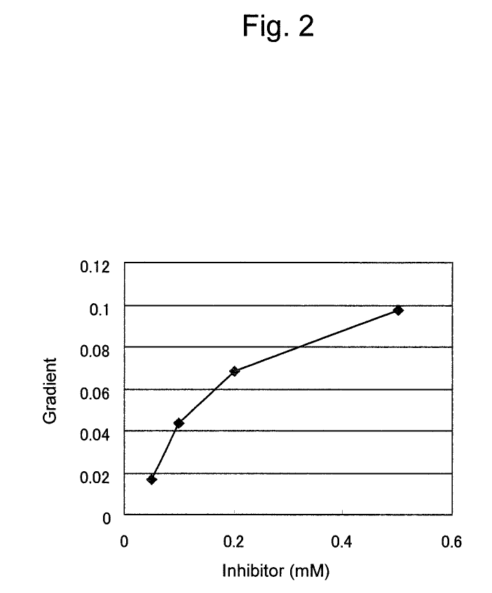

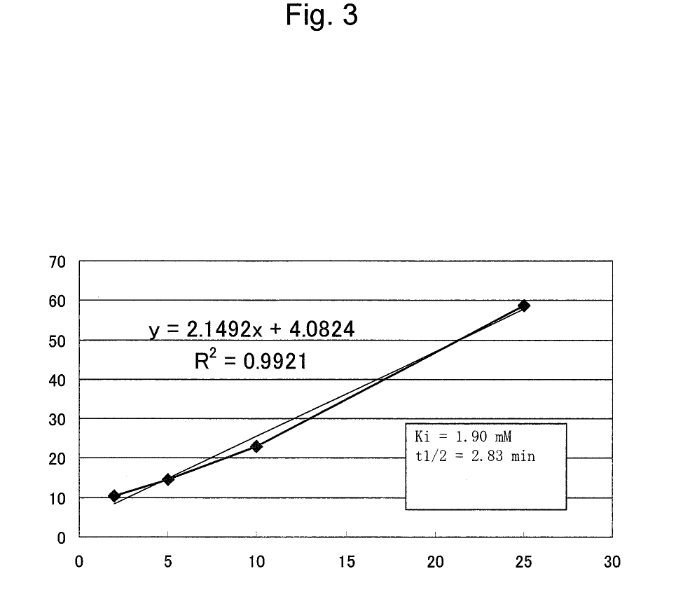

As described below, FIG. 1 shows hydrolysis amounts at glycoside bond site of p-nitrophenyl-α-N-acetyl neuraminic acid after 10-minute pre-incubation. FIG. 2 shows an inhibition curve indicating inhibitory ability of this inhibitor at each use amount (concentration). FIG. 3 shows an inhibition constant (Ki=1.90 mM) and half-life activity (t/_{1/2}=2.83 min) by inhibition, which are calculated from an inhibition curve.

### Example 14

### Comparison of MALDI TOF-MS Spectra before (Upper) and after (Lower) Anti-Dansyl Antibody Column against Protease Degradation Product of Neuraminidase Processed with Inhibitor of the Present Invention

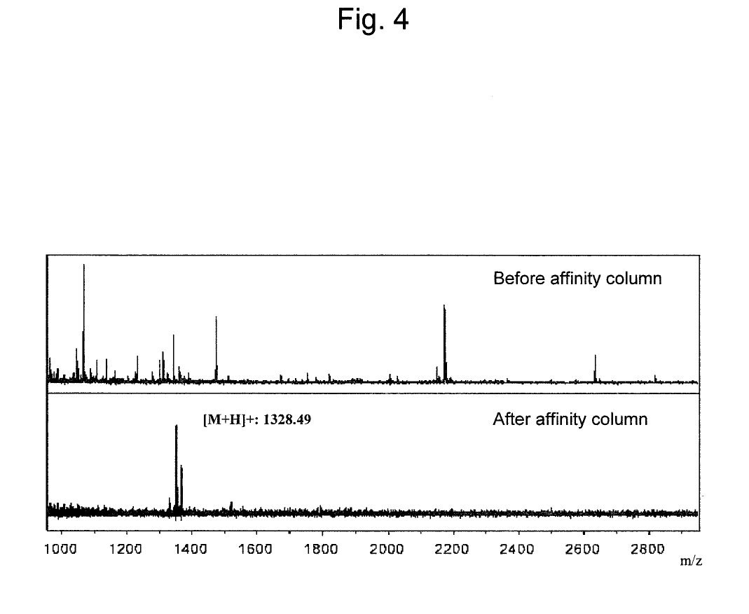

Neuraminidase (50 μg) produced by Vibro-Choralie was incubated for 30 minutes in 50 mM phosphoric acid-tartaric acid buffer (pH 5.0, 100 μl) together with the compound 6 (5 μg). Excessive amount of the compound 6 was isolated from the reaction solution by gel filtration column method (Sephadex G-25), and concentrated by freeze-drying method. Next, the obtained residue was dissolved in 20 mM hydrochloric acid (pH 2.0, 100 μl), and digested into peptide fragments using pepsin (5 μg). The digestive juice was freeze-dried, and peptide fragments having a dansyl derivative, that is an aglycone site of the compound 6, were isolated using an anti-dansyl antibody column. FIG. 4 shows charts of MALDI-TOF-MS analysis before and after processing with the anti-dansyl antibody column. Further, a binding site of aglycone site of the compound 6 against neuraminidase was determined from the MALDI-TOF-MS/MS analysis of the obtained peptide fragments.

### Example 15

### Capture and Isolation of Neuraminidase Using Resin 12 Coated with Neuraminidase Inhibitor, and Sequence Analysis and Attribution of Captured Protein

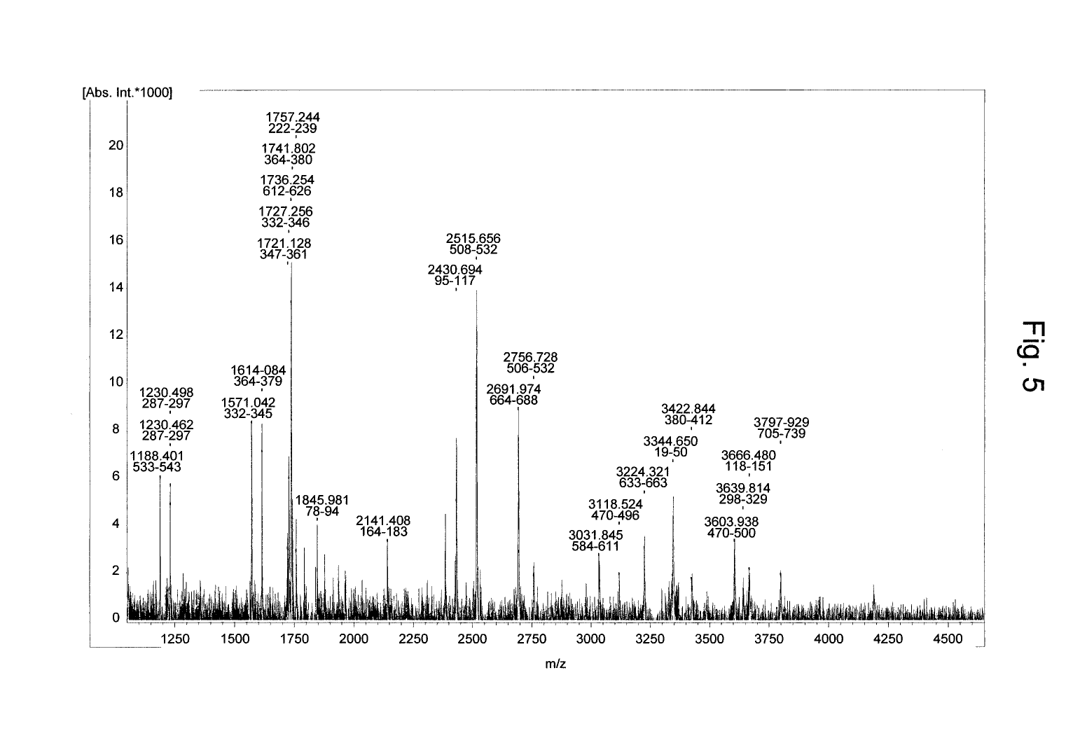

A suspension of the resin 12 coated with neuraminidase inhibitor (100 μl; 2 mg of resin) was added to a culture solution containing neuraminidase (equivalent to 25 μg) produced by Vibro-Choralie, and incubated for one hour. This reaction solution was separated by filtration, and the resin was washed with Milli-Q water, 100 mM guanidine hydrochloride buffer, methanol, and Milli-Q water. This resin was suspended in 20 mM ammonium carbonate buffer (pH 7.8, 100 μl), and digested into peptide fragments using trypsin (5 μg). The reaction solution was filtrated, and sequence analysis of neuraminidase was conducted from MALDI-TOF-MS/MS analysis on peptide fragments in the filtrate. FIG. 5 shows a chart of MALDI-TOF-MS analysis on this filtrate. Further, the kind and attribution of origin of protein were searched by MASCOT score. Neuraminidase produced by Vibro-Choralie was attributable at score 153, and proteins other than that were at 50 or less.

### Example 16

### Preparation of Gold Fine Particle 19 Exhibiting Oxylamino Group

A KAuCl₄ solution (10 mg/ml, 100 μl) and 35% hydrazine aqueous solution (40 μl) were added to a mixture solution of: 10 mM methanol solution of the compound 14 (100 μl); 10 mM aqueous solution of the compound 15 (100 μl); and Milli-Q water (100 μl), followed by stirring for 3 hours. The produced gold fine particles were collected by a centrifugal ultrafiltration tube, and washed with water and methanol. Next, the gold fine particles were suspended in 40% TFA/methanol, and stirred for 2 hours. The solvent was removed by distillation, the residue was suspended in water and concentrated by a centrifugal ultrafiltration tube, and washed with water and methanol, thereby providing the gold fine particles 19. The production of the gold fine particles 19 was confirmed by MALDI-TOF MS. Electron microscopy of the particles 19 found that the particles had an average particle diameter of 1.6 nm.

### Example 17

### Preparation of Gold Fine Particle 20 Exhibiting Neuraminidase Inhibitor

The gold fine particles 19 were suspended in 10 mM aqueous solution (5 μl) of the compound 15 and a Milli-Q water-methanol mixture solution (20 μl, mixture ratio=1:1), followed by stirring overnight. The gold fine particles were collected by a centrifugal ultrafiltration tube, and washed with water and methanol, thereby providing gold fine particles 20. The production of the gold fine particles 20 was confirmed by MALDI-TOF MS.

### Formulation Example 1

A formulation containing the following components is manufactured.

Components

| | |
|---|---|
| Compound represented by formula (I) | 10 mg |
| Lactose | 90 mg |
| Crystalline fine cellulose | 30 mg |
| CMC-Na | 15 mg |
| Magnesium stearate | 5 mg |
| | 150 mg |

A compound represented by the formula (I), lactose, crystalline fine cellulose, CMC-Na (carboxymethylcellulose sodium salt) were sifted with a 60-mesh sieve and mixed with one another. The mixture powder is mixed with magnesium stearate, thus providing a mixture powder for formulation. The mixture powder is compressed to form a tablet of 150 mg.

### Formulation Example 2

The following components are heated, mixed, and sterilized, and then an injection is formulated.

Components

| | | |
|---|---|---|
| Compound represented by formula (I) | 3 | mg |
| Nonionic surfactant | 15 | mg |
| Purified water for injection | 1 | ml |

All publications, patents, and patent applications cited herein are incorporated herein by reference in their entirety.

### INDUSTRIAL APPLICABILITY

A compound of the present invention has irreversible inhibitory activity against neuraminidase, and useful as a therapeutic agent, a preventive agent, or a diagnostic agent for various diseases and infectious diseases involving neuraminidase. Specifically, the compound can be used as a virus detection agent, a microorganism detection agent, a protozoan detection agent, an agent for detecting an inflammatory site, an agent for detecting an oncogenic transformation site, an antiviral agent, an antibacterial agent, an antiprotozoal agent, an anti-inflammatory agent, an anticancer agent. Further, the compound allows a neuraminidase-active site to be arbitrarily labeled, and thus can be used for detecting neuraminidase and determining an active site thereof in the clinical and biochemical research. Furthermore, the compound can be bonded, instead of a marker group, to a physiologically active substance such as an antibiotic or an inorganic compound having antibacterial activity, and thereby the compound can be used as drug delivery system for enabling a drug to be specifically administered to a diseased site actively secreting neuraminidase. Still further, binding of the compound to magnetic particles enables MRI diagnosis on inflammatory and oncogenic transformation sites producing neuraminidase.

1. A compound represented by the following formula (I),
or a salt thereof, or a hydrate thereof, 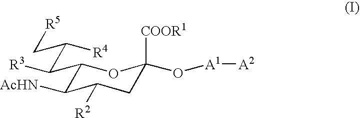
wherein:
A¹ represents an aryl group optionally having a substituent group or a heteroaryl group optionally having a substituent group;
A² represents —CX₂R⁶ or —CHXR⁶ wherein X represents —F, —Cl, —Br, or —I;
R¹ represents a hydrogen atom or an alkyl group optionally having a substituent group;
R², R³, R⁴, and R⁵ represent each independently —OC(═O)R⁶, —OR⁶, —N(R⁶)₂, —N₃, —NHC(═NH)NHR⁶, —NHCOR⁶, —OSO₃R⁶, —OPO₃(R⁶)₂, F, Cl, Br, or I; and
R⁶ represents each independently a hydrogen atom, an alkyl group optionally having a substituent group, an aryl group optionally having a substituent group, or an optionally substituted heteroaryl group.

2. A compound represented by the following formula (II),
or a salt thereof, or a hydrate thereof, 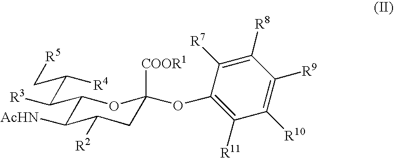
wherein:
R², R², R³, R⁴, and R⁵ are the same as defined in claim 1;
R⁷, R⁸, R⁹, R¹⁰, and R¹¹ represent each independently a hydrogen atom, —NO₂, —F, —Cl, —Br, —I, —N₃, —CN, —Ph, —CX₂R¹², —CHXR¹², —(CH₂)ₘR¹², —CR¹²═CR^{12′}—R^{12″}, —C≡C—R¹², —OP(═O)R¹²(OR^{12′}), —P(═O)R¹²(OR^{12′}), —OPR¹²(OR^{12′}), —PR¹²(OR^{12′}), —OP(R¹²)₂, —P(R¹²)₂, —OS(═O)₂R¹², —S(═O)₂R¹², —OS(═O)R¹², —S(═O)R¹², —C(═O)R¹², or —NR¹²C(═O)R^{12′} (wherein at least one of R⁷, R⁹, and R¹¹ represents each independently —CX₂R¹² or —CHXR¹²);
R¹², R^{12′}, and R^{12″} represent each independently a hydrogen atom, an alkyl group optionally having a substituent group, an aryl group optionally having a substituent group, or a heteroaryl group optionally having a substituent group; and
m represents an integer from 1 to 20,
with the proviso that at least four of R⁷, R⁸, R⁹, R¹⁰ and R¹¹ are not simultaneously a hydrogen atom.

3. A compound represented by the following formula (III), or a salt thereof, or a hydrate thereof, 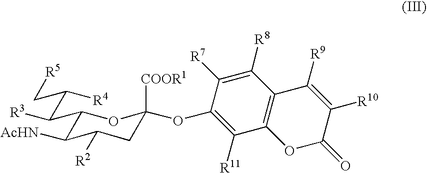
wherein:
R¹, R², R³, R⁴, and R⁵ are the same as defined in (1); and
R⁷, R⁸, R⁹, R¹⁰, and R¹¹ are the same as defined in claim 2.

4. A compound represented by the following formula (IV),
or a salt thereof, or a hydrate thereof, 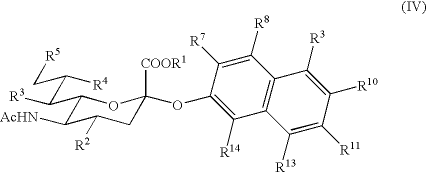
wherein:
R¹, R², R³, R⁴, and R⁵ are the same as defined in claim 1;
R⁷, R⁸, R⁹, R¹⁰, R¹¹, R¹³, and R¹⁴ represent each independently a hydrogen atom, —NO₂, —F, —Cl, —Br, —I, —N₃, —CN, —Ph, —CX₂R¹², —CHXR¹², —(CH₂)ₘR¹², —CR¹²═CR^{12′}—R^{12″}, —C≡C—R¹², —OP(═O)R¹²(OR^{12′}), —P(═O)R¹²(OR^{12′}), —OPR¹²(OR^{12′}), —PR¹²(OR^{12′}), —OP(R¹²)₂, —P(R¹²)₂, —OS(═O)₂R¹², —S(═O)₂R¹², —OS(═O)R¹², —S(═O)R¹², —C(═O)R¹², or —NR¹²C(═O)R^{12′}; and
R¹², R^{12′}, R^{12″}, and m are the same as defined in claim 2.

5. A compound represented by the following formula (V),
or a salt thereof, or a hydrate thereof, 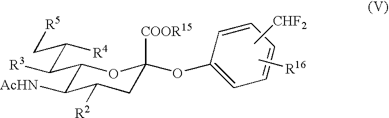
wherein:
R², R³, R⁴ and R⁵ are the same as defined in claim 1;
R¹⁵ represents a hydrogen atom or an alkyl group; and
R¹⁶ represents an alkyl group optionally having a substituent group, an alkenyl group optionally having a substituent group, an alkynyl group optionally having a substituent group, an aryl group optionally having a substituent group, a heteroaryl group optionally having a substituent group, —NO₂, —N₃, an alkyloxy group, an aminooxy group, an alkyloxycarbonyl group, —NHCOR¹⁷ (wherein R¹⁷ represents a hydrogen atom, an alkyl group optionally having a substituent group, an alkenyl group optionally having a substituent group, an alkynyl group optionally having a substituent group, an aryl group optionally having a substituent group, a heteroaryl group optionally having a substituent group, a carboxyl group, an alkyloxycarbonyl group, an amide group, or a marker group), or a marker group.

6. A compound represented by the following formula (VI),
or a salt thereof, or a hydrate thereof, 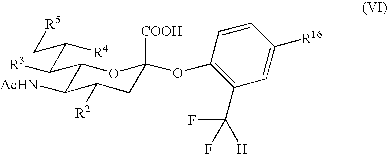
wherein:
R¹, R², R³, R⁴ and R⁵ are the same as defined in claim 1; and
R¹⁶ is the same as defined in claim 5.

7. The compound according to claim 6, wherein R¹⁶ of the formula (VI) is represented by the following formula,
or a salt thereof, or a hydrate thereof, 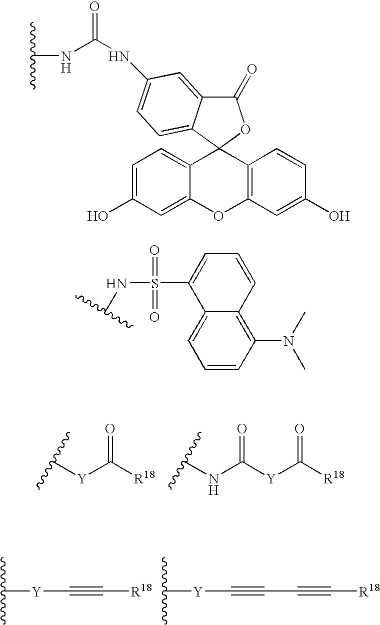
wherein:
Y is selected from the group consisting of —NHCO—, —S—, —O—, —NH—, C₁₋₂₀ alkylene group, C₁₋₂₀ arylene group, and a marker group;
R¹⁸ represents a hydrogen atom, an alkyl group optionally having a substituent group, an alkenyl group optionally having a substituent group, an alkynyl group optionally having a substituent group, an aryl group optionally having a substituent group, a heteroaryl group optionally having a substituent group, —NO₂, an amino group optionally having a substituent group, —N₃, a hydroxyl group, an alkyloxy group, an aminooxy group, an alkyloxycarbonyl group, —NHCOR¹⁷ (wherein R¹⁷ is the same as defined in claim 5), or a marker group.

8. The compound according to any of claims 1 to 7,
or a salt thereof, or a hydrate thereof,
wherein R¹ represents a hydrogen atom or alkyl; R² to R⁵ represent each independently a hydroxyl group, an acyloxy group, an amino group, or a guanidyl group.

9. The compound according to any of claims 1 to 8,
or a salt thereof, or a hydrate thereof,
wherein R² is an amino group or a guanidyl group.

10. A compound obtained by reacting the compound according to any of claims 1 to 9 with a carrier containing a functional group selected from the group consisting of an optionally protected aminooxy group, N-alkylaminooxy group, hydrazide group, azide group, thiosemicarbazide group, 1,2-dithiol group, alkyl iodide group, acid anhydride, acid fluoride, acid chloride, acid bromide, tetrafluorophenyl ether group, N-hydroxysuccinimide ether group, haloalkyl group, haloacyl group, amino group, hydroxyl group, aldehyde group, acetylene group, ketone group, carboxyl group, and cysteine residue.

11. The compound according to claim 10, which is selected from the group of polymers or copolymers of:
a) acrylamides, methacrylamides, acrylic acids, methacrylic acids, styrenes, or fatty acid vinylesters;
b) silica carriers, resin carriers, magnetic beads, or metal carriers;
c) compounds represented by the following formulas: 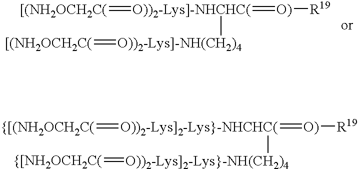
[NH₂OCH₂C(═O))₂—Lys]—NHCHC(═O)—R¹⁹[(NH₂OCH₂C(═O))₂—Lys]—NH(CH₂¹)₄
{[(NH₂OCH₂C(═O))₂—Lys]₂—Lys}—NHCHC(═O)—R¹⁹{[(NH₂OCH₂C(═O))₂—Lys]₂—Lys}—NH(CH₂¹)₄
wherein R¹⁹ represents a hydroxyl group or an amino group, Lys represents lysine, and Cys represents cysteine, 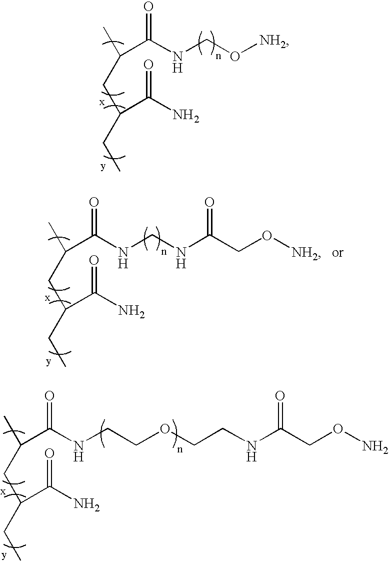
wherein n represents an integer from 1 to 15, and x:y is 1:0 to 1:1000; and
d) compounds represented by the following formula, 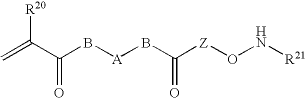
wherein A represents a C₂₋₁₂ alkylene group optionally having a substituent group, B represents —NH— or —O—, R²⁰ represents a hydrogen atom or a methyl group, Z represents a C₁₋₅ alkylene group optionally having a substituent group, and R²¹ represents a hydrogen atom or a protecting group of amino group.

12. The compound according to any of claims 1 to 11, having covalent bond with a physiologically active substance.

13. The compound according to any of claims 1 to 12 having covalent bond or coordinate bond with silver.

14. The compound according to any of claims 1 to 13, having covalent bond or coordinate bond with a magnetic substance.

15. A drug comprising the compound according to any of claims 1 to 14.

16. A therapeutic agent for an infectious disease comprising the compound according to any of claims 1 to 14.

17. A therapeutic agent for an infectious disease involving neuraminidase comprising the compound according to any of claims 1 to 14.

18. An agent for detecting neuraminidase-producing microorganisms comprising the compound according to any of claims 1 to 14.

19. An agent for detecting a neuraminidase-expressing site comprising the compound according to any of claims 1 to 14.

20. An agent for isolating and detecting neuraminidase comprising the compound according to any of claims 1 to 14.

21. A neuraminidase inhibitor comprising the compound according to any of claims 1 to 14.

22. A neuraminidase analysis method comprising the steps of: adding the compound according to any of clams 1 to 14 to a solution containing neuraminidase to capture neuraminidase; adding protease to the solution to digest neuraminidase into peptide fragments; and then analyzing the peptide fragments by mass spectrometry.

23. A protein analysis method comprising the steps of: adding the compound according to any of claims 1 to 14 to a solution containing neuraminidase to capture neuraminidase; and analyzing proteins bonded to the neuraminidase by ELISA method, fluorescence or staining method.

24. A disease diagnostic method which comprises the steps of: adding the compound according to any of claims 1 to 14 to a solution or suspension containing biological specimens, bacteria and/or viruses; capturing biological specimens, bacteria and/or viruses containing neuraminidase; then performing antigenic analysis for the captured biological specimens, bacteria and/or viruses using antibodies or analyzing DNA or RNA using PCR.
